(19) United States
(12) Patent Application Publication
(10) Pub. No.: US 2025/0216398 A1
(43) Pub. Date: Jul. 3, 2025

(54) IDENTIFICATION OF AMINO ACIDS OR SHORT PEPTIDES

(71) Applicant: LUXEMBOURG INSTITUTE OF SCIENCE AND TECHNOLOGY (LIST), Eschsur- Alzette (LU)

(72) Inventor: Cesar PASCUAL GARCIA, Esch sur Alzette (LU)

(21) Appl. No.: 18/847,426

(22) PCT Filed: Mar. 13, 2023
(86) PCT No.: PCT/EP2023/056332

§ 371 Date: Sep. 16, 2024

(30) Foreign Application Priority Data
Mar. 15, 2022 (LU) LU501669

(51) Int. Cl. G01N 33/68; G01N 27/22; G01N 27/414; G01N 33/84

(52) U.S. Cl. CPC G01N 33/6818; G01N 27/221; G01N 27/4145; G01N 33/84

(57) ABSTRACT

An aspect of the invention relates to a method for identifying an amino acid is proposed. The method comprises immobilising an amino acid on the surface of a FET sensor, acquiring a fingerprint of the immobilized amino acid, the acquisition of the fingerprint including measuring at least one of the surface potential and the gate capacitance of the FET sensor with the amino acid immobilized thereon as a function of pH, and looking up the acquired fingerprint in a fingerprint database. Further aspects of the invention relate to a device for recording a fingerprint of an analyte and a method for sequencing a peptide.

### BACKGROUND OF THE INVENTION

The invention generally relates to identification of amino acids, i.e., the determination of the amino acid species to which an a priori unknown amino acid belongs. In a further aspect, the invention also relates to the sequencing of peptides.

Proteomics is considered as the next frontier in medicine and green industries. It promises to enable the creation of enzymes for green industries or personalised biomarkers and drugs for individually tailored medicine. However, current analytical methods based on mass spectrometry are not adequate to translate biological discoveries into daily applications. What is considered necessary is accessing the high-resolution protein information of custom-made assays, using limited sample quantities and providing fast results. New approaches based on nanopores or the imaging of single proteins subjected to Edman degradation raised hopes of lowering both detection limits and sequencing costs. However, chemically controlling and manipulating minimal quantities of peptides, the identification of amino acids and finding which peptide sequences are relevant within the 3D structure of proteins remain yet unsolved issues of these new technologies. Today, Mass Spectroscopy (MS) is the gold standard allowing analyses within hours. However, MS requires costly instrumentation, excessively large quantities of samples and has low modularity to combine with other assays that study protein interactions.

There are several reasons why advances in proteomics have not taken place at the same pace as in genomics:
a. In the absence of molecular amplification (like the polymerase chain reaction for DNA), there is a need for other chemical manipulation methods able to parallelise the analysis of peptides while working with low quantities.
b. The read-out of amino acids is still based on cumbersome slow approaches like Blots or expensive approaches like MS that hamper the implementation of in-situ identification.
c. The functionality of proteins depends not only on the primary structure determined by the sequence, but also on the 3D structure and its ability to interact with (bind to) other molecules.

To bypass MS techniques of transduction similar to the ones used in DNA sequencing have been proposed for amino acid recognition. Nanopores have been suggested to transduce the electrostatic signal of amino acids in polypeptide strings. However, it is more difficult to design a driving force to make the peptide pass through the nanopore, as amino acids may have different charges. Also, identifying 20 (canonical) amino acids is much more challenging than only four nucleotides. Recently, an approach based on Edman degradation and the optical labelling of two amino acids gained attention because it was able to identify single proteins from zeptomolar concentrations analysing the frequency of cysteine and lysine (J. Swaminathan et al., “Highly parallel single-molecule identification of proteins in zeptomole-scale mixtures,” Nat. Biotechnol., vol. 36, no. 11, pp. 1076-1082, 2018). However, the optical labelling of 20 AAs seems unfeasible due to the chemical equivalences among them. The hazardous chemistry used and the limited multiplexing capabilities of this method also limit this approach.

WO 2021/211631 A2 relates single molecule N-terminal sequencing using electrical signals. A method for polypeptide sequencing is proposed, wherein a polypeptide is immobilized on FET sensor, an amino acid is removed from the polypeptide and the sensor is used to measure a charge, conductivity, or impedance, or change thereof, in the solution subsequent to removal of the amino acid from the polypeptide. The removal of the terminal amino acid may be effected by Edman degradation. WO 2021/211631 A2 mentions that non-optical sequencing of peptides may rely on a “spectral or electromagnetic signature”. Peptides may be labelled with an electrical or spectrally relevant label or may use no label at all. The labelled or non labelled peptides are diluted and coupled onto an electrically active and addressable surface having FETs. The labelled or non-labelled peptides are directed to the electrically active surface with a bond such that there is one peptide per sensor. The document that mentions that each addressable sensor is electrically stimulated, and an electrical or spectral signature recorded due to the adjacent attached peptide. Peptide degradation is then subsequently performed over the surface whereby the entire collection of peptides loses one amino acid and any label that was affixed to it; thus, potentially changing the local spectroscopic signal. The spectral signatures over the addressable units between peptide degradation cycles are collected and the spectral changes that occur from each recording device are analyzed to determine the set of possible peptides each spectral signature corresponds. A spectral analysis is used to estimate the concentrations of the individual species of peptide present in the original mixture. WO 2021/211631 A2 details neither how the sensors are electrically stimulated nor how the “electrical or spectral signatures” are recorded.

WO 2004/059283 A2 discloses an apparatus and method for Edman degradation using a microfluidic system to identify and characterize peptides. The method comprises cleaving the terminal amino acid of a peptide, and separating the cleaved amino acid from the microfluidic device. The cleavage product is a single cleaved amino acid produced from a single cycle of an Edman degradation. The cleavage product is sent to a downstream separation module which isolates the single cleaved amino acid.

U.S. Pat. No. 6,677,114 B1 uses a combination of electrophoretic methods conducted in series to resolve mixtures of proteins. The series of electrophoretic methods are typically conducted in such a way that proteins in an applied sample for each electrophoretic method of the series are isolated or resolved physically, temporally or spatially to form a plurality of fractions each of which include only a subset of proteins of the applied sample. To obtain additional information, such as molecular weight and partial sequence, fractions collected from the final electrophoretic method may be individually analyzed by mass spectroscopy.

US 2018/0372752 A1 describes methods for sequencing polypeptides. The method comprises affixing a polypeptide to a substrate and contacting the polypeptide with a plurality of probes. Each probe selectively binds to an N-terminal amino acid or an N-terminal amino acid derivative. Probes bound to the polypeptide molecule are then identified before cleaving the N-terminal amino acid or N-terminal amino acid derivative of the polypeptide. Also provided are methods for the sequencing a plurality of polypeptide molecules in a sample and probes specific for N-terminal amino acids or N-terminal amino acid derivatives.

EP 3 013 983 A1 provides methods and assay systems for use in spatially encoded biological assays, including assays to determine a spatial pattern of abundance, expression, and/or activity of one or more biological targets across multiple sites in a sample. In particular, the present disclosure provides methods and assay systems capable of high levels of multiplexing where reagents are provided to a biological sample in order to address tag the sites to which reagents are delivered; instrumentation capable of controlled delivery of reagents, in particular, microfluidic device-based instrumentation; and a decoding scheme providing a readout that is digital in nature.

The paper “Subfemtomole level protein sequencing by Edman degradation carried out in a microfluidic chip”, Chem. Commun., 2007, 2488-2490, discloses a microfluidic chip based Edman degradation system, in which Edman degradation can be carried out with peptide at a subfemtomole level. The proposed system immobilizes the peptide on a reaction cartridge before PITC is delivered to the reaction cartridge. The coupling reaction is initiated using TMA vapor. Thereafter, TFA vapor is used to initiate the cleavage reaction. The cleaved amino acid is then spotted onto the target for MALDI-TOF-MS.

### SUMMARY OF THE INVENTION

The present invention proposes to use a field-effect transistor (FET) to study charge variations of peptides and amino acids, to record unique fingerprints which allow identification of amino acids and peptides.

As used herein, the expression “peptide” designates a biomolecule formed by a chain of amino acids linked by peptide bonds. The expression peptide may encompass short amino acid chains, such as, e.g., dipeptides, tripeptides, tetrapeptides, or oligopeptides (comprising up to 20 consecutive amino acids), as well as polypeptides (comprising more than 20 consecutive amino acids). Polypeptides comprising more than about 50 consecutive amino acids linked by peptide bonds may be referred to as “proteins”. Hereinafter, the expression “short peptide” will be used to designate a peptide comprising up to 10 consecutive amino acids linked by peptide bonds, e.g., up to 2, 3, 4, 5, 6, 7, 8 or 9 consecutive amino acids linked by peptide bonds.

As used herein, the term “amino acid” includes the “neutral” form (amino group unprotonated and carboxyl group undissociated) as well as the possible amino-acid residues that form when two or more amino acids combine to form a peptide. Such residues may lack a hydrogen atom of the amino group the hydroxyl moiety of the carboxyl group or both. The units of a peptide are, therefore, amino-acid residues but they may be referred to herein as “amino acids”, which is also common practice in the literature.

According to a first aspect of the invention, a method for identifying an amino acid is proposed. The method comprises immobilising an amino acid on the surface of a FET sensor, acquiring a fingerprint of the immobilized amino acid, the acquisition of the fingerprint including measuring at least one of the surface potential and the gate capacitance of the FET sensor with the amino acid immobilized thereon as a function of pH (of the bulk electrolyte), and looking up the acquired fingerprint in a fingerprint database. A FET sensor uses the solution (containing the amino acid or peptide to be identified) adjacent the sensor surface as the gate electrode. As used herein, “pH” may be defined as the negative logarithm (to base 10) of the hydrogen ion concentration (pH=−log₁₀[H+]) or as the negative logarithm (to base 10) of the hydrogen ion activity (pH=−log₁₀(a_{H+})). If an organic electrolyte should be used, “pH” may be defined as the negative logarithm (to base 10) of the (equivalent) concentration of protons or protonated species (sometimes referred to as pH*). What matters is that the same definition is used for the acquired fingerprint as for the fingerprints in the database to which the acquired fingerprint is compared.

In the present context, the term “fingerprint” designates a set of uniquely identifying data, the acquisition of which permits to identify an individual species or a subgroup of amino acid species. The fingerprint is extracted from the surface potential and/or the gate capacitance of the FET sensor with the amino acid immobilized thereon, measured as a function of pH (i.e., for different pH values, the surface potential and/or the gate capacitance of the FET sensor with the amino acid immobilized thereon). Preferably, the measurement of the surface potential and/or the gate capacitance is carried out at least over the full pH range from 3 to 10, more preferably from 2 to 11, still more preferably from 1 to 11, yet more preferably from 1 to 12 and most preferably from 0 to 14 or even higher (pH values higher than 14 can be reached by using organic solvent(s) as the electrolyte, e.g., acetonitrile). Preferably, a data point is taken at pH increments or decrements of 0.2 or less, more preferably at pH in increments or decrements of 0.1 or less, still more preferably at pH in increments or decrements of 0.05 or less and even still more preferably at pH in increments or decrements of 0.01 or less, e.g., of 0.005 or less, or of 0.001 or less.

Charge and current of impedance were found not to allow differentiating between different amino acid species. However, amino acids are amphoteric molecules, i.e., they change between different protonation states depending on the acidity of their environment. Each amino acid has a characteristic set of pKₐ values (pKₐ=−log₁₀(Kₐ), where Kₐ is the acid dissociation constant). An amino acid in solution may be subjected to a titration, e.g., starting in acidic conditions (low pH) and adding a strong base. The pH increases nonlinearly with the amount of added base: when the pH is close to a pKₐ value of the amino acid, the titration curve becomes flat as the amino acid buffers the addition of base. Steeper increases of the pH occur at the equivalence points (where the added base completely neutralises an acidic proton released from the amino acid).

The present invention relies on the surprising observation that, when an amino acid is immobilized on a FET sensor, the changes of protonation states occurring when the pH of the bulk solution is varied change the surface potential and the gate capacitance of the FET sensor, which can be measured. In the article by P. Bergveld, R. E. G. van Hal and J. C. T. Eijkel, “The remarkable similarity between the acid-base properties of ISFETs and proteins and the consequences for the design of ISFET biosensors”, Biosensors & Bioelectronics 10 (1995), pp. 405-414, the authors expressed the opinion that any externally induced modulation of the surface potential and the pH directly at the ISFET surface (pHₛ) will not be measured by the ISFET and that it was not expected that a useful sensor could be developed in this way.

The amino acid immobilized on the FET sensing surface may be an individual (single) amino acid. Alternatively, the amino acid immobilized on the FET sensing surface could be part of a short peptide, in which case the fingerprint is a fingerprint of the immobilized amino acid as part of the short peptide, i.e., a fingerprint of the short peptide itself. Specifically, the method may thus be for identifying a short peptide and comprise immobilising the short peptide on the surface of the FET sensor, acquiring a fingerprint of the immobilized short peptide, the acquisition of the fingerprint including measuring at least one of the surface potential and the gate capacitance of the FET sensor with the short peptide immobilized thereon as a function of pH of the bulk electrolyte, and looking up the acquired fingerprint in a fingerprint database.

Different methods for the surface potential measurement are described in literature and need not be discussed in detail. The surface potential can, e.g., be deduced from the output current, or more classically, from the compensation with a reference electrode. The canonical method for measuring the surface potential includes measuring the threshold voltage of the FET at each pH. However, for practical reasons, one may also fix a source-drain voltage with a small, but measurable current in the linear range of the transistor channel, and compensate the reference voltage V_{ref} with the pH to keep the current constant. The gate capacitance can be obtained by electrical impedance spectroscopy from the transconductance of the FET sensor measured at different frequencies. The gate capacitance results from different contributions in series, of a constant device capacitance, a contribution from the peptide, and a double layer contribution from the electrolyte. The device capacitance corresponds to the dielectric layer separating the transistor channel from the molecules and the electrolyte. The device capacitance being constant (and thus not dependent upon the attached species), it does not contribute to the fingerprints. In some novel devices like graphene-FETs, the molecules and the electrolyte are directly coupled to the transistor channel by the Dirac point, and this capacitance does not have any significant contribution. Amino acids and peptides have different dielectric constants and lengths, which contribute to the fingerprints. Moreover, the amphoteric properties of amino acids changing charge depending on the pH also contribute to the charging of the electrolyte creating the double layer capacitance. As this capacitance also depends on the different pKa of the amino acids, it also contributes to the fingerprints. The series combination of the peptide and the double layer capacitances may be defined as surface capacitance and the series combination of the surface capacitance and any constant device capacitance may be defined as the gate capacitance.

The FET sensor may be arranged in a reaction chamber of a reaction cell configured as an electrochemical transducer translating an applied voltage or current into a change of pH within the reaction chamber. Measuring the surface potential and/or the gate capacitance of the FET sensor with the amino acid immobilized thereon as a function of pH may include electrochemically varying the pH in the reaction chamber. A suitable reactor for modifying the pH is described in WO 2017/108796A1, incorporated herein by reference in its entirety. Another reactor for modifying the pH is described in Sensors and Actuators B: Chemical, Vol. 47, Issues 1-3, 30 Apr. 1998, pp. 48-53: “A flow-through cell with integrated coulometric pH actuator”, Böhm S. et al.

During the measurement of the surface potential and/or the gate capacitance as a function of pH, the reaction cell may be closed, the volume of the reaction cell thus being confined to less than 10 nl (nanoliter), preferably to less than 5 nl, more preferably to less than 3 nl, still more preferably to less than 2 nl and even more preferably to less than 1 nl.

The acquisition of the fingerprint may include measuring both the surface potential and the gate capacitance of the FET sensor with the amino acid immobilized thereon as a function of pH.

The acquisition of the fingerprint may include measuring the surface potential and/or the gate capacitance of the FET sensor with the amino acid immobilized thereon as a function of pH at constant temperature. More preferably, the acquisition of the fingerprint may include measuring the surface potential and/or the gate capacitance of the FET sensor with the amino acid immobilized thereon as a function of pH at at least two (different) temperatures. In this case the temperature would preferably be maintained constant during each measurement of the surface potential and/or the gate capacitance as a function of pH.

It should be noted that the curves representing the surface potential and/or the gate capacitance of the FET sensor with the amino acid immobilized thereon as a function of pH need not be themselves the fingerprints. The fingerprint could be derived from the measured curves. For instance, the acquisition of the fingerprint could comprise determining the second derivative of the surface potential with respect to pH. It was indeed found that the surface potential may appear to be a linear (affine) function of the pH and characteristic fingerprints may only become apparent after a double differentiation. Additionally, or alternatively, the acquisition of the fingerprint may comprise determining the first derivative of the gate capacitance with respect to pH.

The amino acid could be immobilized on the surface of the FET sensor with a PITC (phenylisothiocyanate, Edman's reagent) homologue reagent. Such reagent could comprise, e.g., phenyl 4-chloro isothiocyanate, or 4-chloro-3(trifluoromethyl)phenyl isothiocyanate.

The FET sensor could comprise a graphene FET sensor. Alternatively, the FET sensor could comprise a semiconductor-based FET (e.g., a Si-based FET) having a dielectric layer made of a high-K dielectric oxide (i.e. having a dielectric constant, κ, equal to or higher than that of Al₂O₃), e.g., Al₂O₃, HfO₂, or TiO₂, as the dielectric layer for the gate.

The FET sensor could include a FinFET, a junction-less FET, or a 2D (also: monolayer) FET, e.g., a 2D molybdenum disulfide FET (monolayer MoS₂ FET).

The fingerprint could be acquired with the FET sensor immersed in a first electrolyte (e.g., water) and a further fingerprint could be acquired with the FET sensor immersed in a second, different electrolyte (the second electrolyte could be organic-solvent-based, e.g., acetonitrile). The combination of both fingerprints may allow removing ambiguities (between amino acid species yielding similar or identical fingerprints in certain electrolytes). Using an electrolyte with a pKa substantially higher than the pKa of water indeed would make it possible to access more charging groups (e.g., the imide groups obtained by formation of the PITC-amino acid).

A second aspect of the invention relates to a method for sequencing a peptide. This method includes identification of amino acids according to the first aspect of the invention as detailed above. Specifically, the method for sequencing a peptide comprises:
sequentially removing amino acids from a (one) terminus of the peptide, the terminus being the N-terminus or the C-terminus of the peptide;
immobilising the amino acids on the surface of a series of FET sensors, each FET sensor of the series corresponding to a known position in the sequence of removal;
acquiring a fingerprint of each immobilized amino acid, the acquisition of the fingerprint including measuring the surface potential and/or the gate capacitance of the respective FET sensor with the amino acid immobilized thereon as a function of pH, and o for each FET sensor, looking up the acquired fingerprint in a fingerprint database.

The peptide to be sequenced is preferably provided in a highly pure form. For instance, it could be sourced from liquid chromatography (LC), high-performance liquid chromatography (HPLC) or another technique capable of isolating the peptide species of interest.

A third aspect of the invention relates to a device for recording a fingerprint of an analyte (a substance of interest), e.g., an amino acid. The device comprises:
a reaction cell configured as an electrochemical transducer translating an applied voltage or current into a change of pH within a reaction chamber of the reaction cell, the reaction chamber having arranged therein an FET sensor, the FET sensor comprising a sensing surface for immobilizing the analyte thereon;
a controller operatively connected to the reaction cell for controlling the pH in the reaction chamber and to the FET sensor for measuring at least one of the surface potential and the gate capacitance of the FET sensor; the controller being configured to execute a fingerprint acquisition routine, which includes recording the at least one of the surface potential and the gate capacitance of the FET sensor while increasing or decreasing the pH in the reaction chamber.

The reaction cell may comprise a heating element in the reaction chamber and the controller may be operatively connected to the heating element for controlling the temperature in the reaction chamber.

The FET sensor could be configured as described above.

In the present document, the verb “to comprise” and the expression “to be comprised of” are used as open transitional phrases meaning “to include” or “to consist at least of”. Unless otherwise implied by context, the use of singular word form is intended to encompass the plural, except when the cardinal number “one” is used: “one” herein means “exactly one”. Ordinal numbers (“first”, “second”, etc.) are used herein to differentiate between different instances of a generic object; no particular order, importance or hierarchy is intended to be implied by the use of these expressions. Furthermore, when plural instances of an object are referred to by ordinal numbers, this does not necessarily mean that no other instances of that object are present (unless this follows clearly from context). When this description refers to “an embodiment”, “one embodiment”, “embodiments”, etc., this means that the features of those embodiments can be used in the combination explicitly presented but also that the features can be combined across embodiments without departing from the invention, unless it follows from context that features cannot be combined.

### BRIEF DESCRIPTION OF THE DRAWINGS

By way of example, preferred, non-limiting embodiments of the invention will now be described in detail with reference to the accompanying drawings, in which:

FIG. 1: is a diagram showing the second derivative, with respect to pH, of the surface potential of a FET sensor having amino acids of the glutamate family attached to the sensing surface thereof;

FIG. 2: is a diagram showing the second derivative, with respect to pH, of the surface potential of a FET sensor having amino acids of the aspartate family attached to the sensing surface thereof;

FIG. 3: is a diagram showing the second derivative, with respect to pH, of the surface potential of a FET sensor having different amino acids attached to the sensing surface thereof;

FIG. 4: is a diagram showing the second derivative, with respect to pH, of the surface potential of a FET sensor having amino acids of the serine family attached to the sensing surface thereof;

FIG. 5: is a set of two diagrams showing, on the left-hand side, the second derivative, with respect to pH, of the surface potential, and on the right-hand side, the total (gate) capacitance (CT) of a FET sensor having amino acids of the pyruvate family attached to the sensing surface thereof;

FIG. 6: is a diagram showing the second derivative, with respect to pH, of the surface potential of a FET sensor having amino acids of the Shikimate family attached to the sensing surface thereof;

FIG. 7: is a schematic exploded perspective view of a reaction cell that may be used in the context of the invention;

FIG. 8: is a schematic top view of a microfluidic device including a plurality of reaction cells as shown in FIG. 7;

FIG. 9: is a schematic illustration of the microfluidic device of FIG. 8 when the reaction cells are open;

FIG. 10: is a schematic illustration of the microfluidic device of FIG. 8 when the reaction cells are closed;

FIG. 11: is a schematic illustration of a peptide sequencing process using the microfluidic device of FIG. 8, different steps of the process being illustrated in FIGS. 11 (A)-(G);

FIG. 12: is a combined diagram showing, on the left-hand side, the second derivative, with respect to pH, of the surface potential and, on the right-hand side, the total gate capacitance (C_{T}) of a FET sensor having various amino acids attached by their C-terminus to the FET sensing surface;

FIG. 13: is a combined diagram showing, on the left-hand side, the second derivative, with respect to pH, of the surface potential and, on the right-hand side, the total gate capacitance (C_{T}) of a FET sensor having various amino acids attached by their N-terminus to the FET sensing surface.

### DETAILED DESCRIPTION OF PREFERRED EMBODIMENTS

Distinguishing be it only the 20 canonical amino acids is much more challenging than distinguishing four nucleotides (with the bases adenine (A), cytosine (C), guanine (G), and thymine (T)). In the case of nucleotides, using a polymerase reaction, there is a selectivity amongst them (A binds to T and G binds to C), which can be used straightforwardly for labelling (e.g., illumina, or ion-torrent technologies use this). Amino acids have chemical equivalents that make selective labelling very difficult. Only cysteine with its thiol side chain has a very distinguishable radical that can be used for selective labelling, and indeed, it has been used to identify proteins with fluorescence proteins and Edman degradation (cf. L. Tang, “Next-generation peptide sequencing,” Nat. Methods, vol. 15, no. 12, p. 997, 2018). Also, for nucleotides, there are only four with different coulomb blockade signals, which makes their recognition with a nanopore (Oxford nanopore) possible. This concept cannot easily be applied on amino acids because it then requires nanopore specifications that are beyond current state of the art technology (sub-nanometer pores that would need to be produced in a controlled way with always the same dimensions and small tolerances).

For the recognition of amino acids with sensors is not a priori clear what can be the transduced signal. Electrical charge sensors (sensing total charge, current or impedance) cannot distinguish amino acids as these carry similar charges. The overall charge of amino acids ranges between −3 and +2 elementary charges, depending on the pH. Identification of peptides using their charge is even more difficult because of the numerous candidate amino acid combinations that could explain the observed charge. Even measuring the charge of a peptide alongside plural Edman degradation cycles (as suggested in above-mentioned WO 2021/211631 A2) will not give rise to a usable fingerprint allowing identification. On top of that, it is not clear how to measure the charge of a peptide and the concentration at the same time: two amino acids with elementary charge +1 produce the same signal as one amino acid with elementary charge +2. Similar issues are encountered when attempting to use optical signals for amino acid identification: amino acids have similar refractive indices and Raman peaks, no specific fluorescence.

For the above reasons, identification of amino acids does not seem feasible with only optical or electrostatic transduction. The identification method proposed herein uses the physicochemical properties of amino acids and peptides. As indicated above, amino acids and peptides have a net charge that depends on acidity, since the amino and carboxylic groups and some of the radicals can have different protonation states depending on the pH. The charge of individual amino acids as a function of pH (which can be determined by an acid titration) provides a fingerprint (of the amino acids in solution).

The present method proposes to identify an amino acid (or a short peptide) immobilized on a surface, in particular the sensing surface (gate) of a FET sensor. Acquiring a fingerprint of the immobilized amino acid (or short peptide) includes measuring the surface potential and/or the gate capacitance of the FET sensor as a function of pH, while the amino acid (or short peptide) is attached to the sensing surface. The acquired fingerprint may then be looked up in a fingerprint database. Pattern recognition algorithms, classifiers and/or artificial intelligence may be used to look up corresponding fingerprints in the database.

When an amino acid is immobilized on a FET sensor, the changes of protonation states occurring when the pH of the bulk solution is varied change the surface potential of the FET sensor, which can be measured.

The signal of the FET sensor depends on the intrinsic buffering capacitance (determined by the chemical species on the surface of the FET sensor) and the double layer capacitance:

$$\frac{\partial {ψ}_{0}}{\partial pH} = - 2.303 \frac{{k}_{B} T}{q} α$$

where Ψ₀ is the surface potential, pH is the pH in the bulk of the electrolyte, KB is the Boltzmann constant, T is the temperature (in K), and q is the elementary charge, respectively. For reference, see P. Bergveld, R. E. G. van Hal and J. C. T. Eijkel, “The remarkable similarity between the acid-base properties of ISFETs and proteins and the consequences for the design of ISFET biosensors”, Biosensors & Bioelectronics 10 (1995), pp. 405-414. The sensitivity parameter a depends on the chemical buffering capacity of the dielectric surface in contact with the electrolyte and the response of the ions in solution that will create the double layer capacitance. Often, α is considered only in the linear range of the FET sensor.

The surface potential Ψ₀ charges the surface capacitance, which may be modelized according to the Gouy-Chapman-Stern (GCS) theory as the Stern layer capacitance C_{Stern} in series with the diffuse-layer capacitance C_{dl}:

$${C}_{GCS}^{- 1} = {C}_{Stern}^{- 1} + {C}_{d l}^{- 1}$$

where C_{GCS} is the double layer capacitance modelized according to the Gouy-Chapman-Stern (GCS) theory.

The sensitive parameter α may be expressed as:

$$α = {( 1 + \frac{2.303 {k}_{B} T {C}_{G C S}}{{q}^{2} {β}_{s}} )}^{- 1}$$

where βₛ is the intrinsic buffer capacity of the sensor surface, which depends on the number of binding sites Nₛ on the sensing surface and their corresponding proton affinities pKa and pKb.

To take into account the amino acid or peptide attached to the sensing surface, the Gouy-Chapman-Stern model may be modified, and the surface capacitance Cₛ be modelized as follows:

$${C}_{s}^{- 1} = {C}_{GCS}^{- 1} + {C}_{peptide}^{- 1}$$

where C_{peptide} is the capacitance of the amino acid or peptide, which in this model is considered to appear in series with C_{GCS}. The total (gate) capacitance may be defined as the series combination of the surface capacitance and any device capacitance (e.g., oxide layer capacitance) that contributes to all fingerprints in the same way. For simplicity, the total (gate) capacitance represented in the drawings disregards such device capacitance and thus corresponds to the surface capacitance

Amino acid fingerprints were found when a FET sensor was functionalised with individual species of amino acids and the surface potential Ψ₀ was measured while the pH (of the bulk electrolyte) was titrated. Due to the action of the double layer capacitance, these fingerprints were not obvious: the surface potential appeared to be linear as a function of pH. However, after a double differentiation of the surface potential, the amino acid fingerprints became apparent.

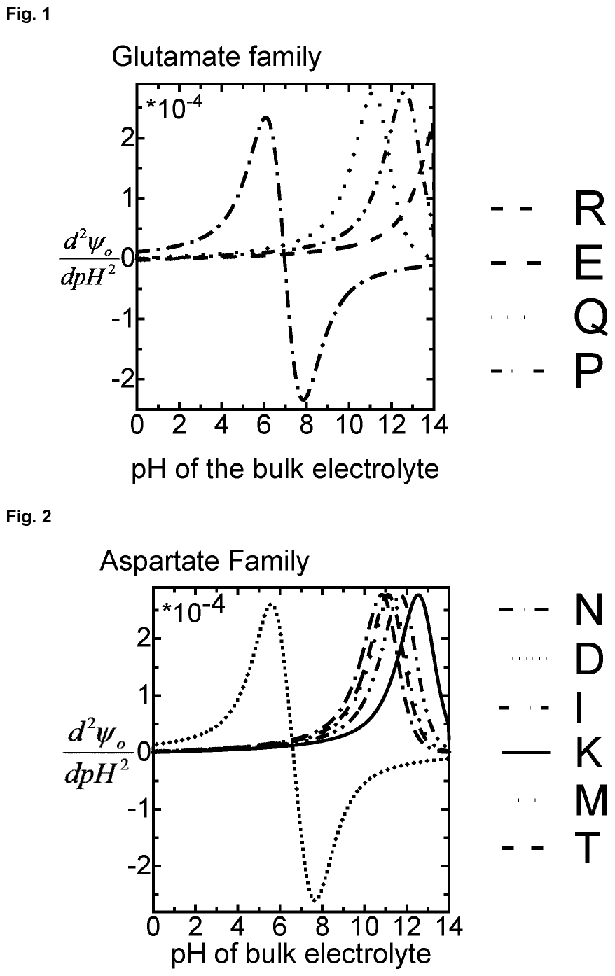

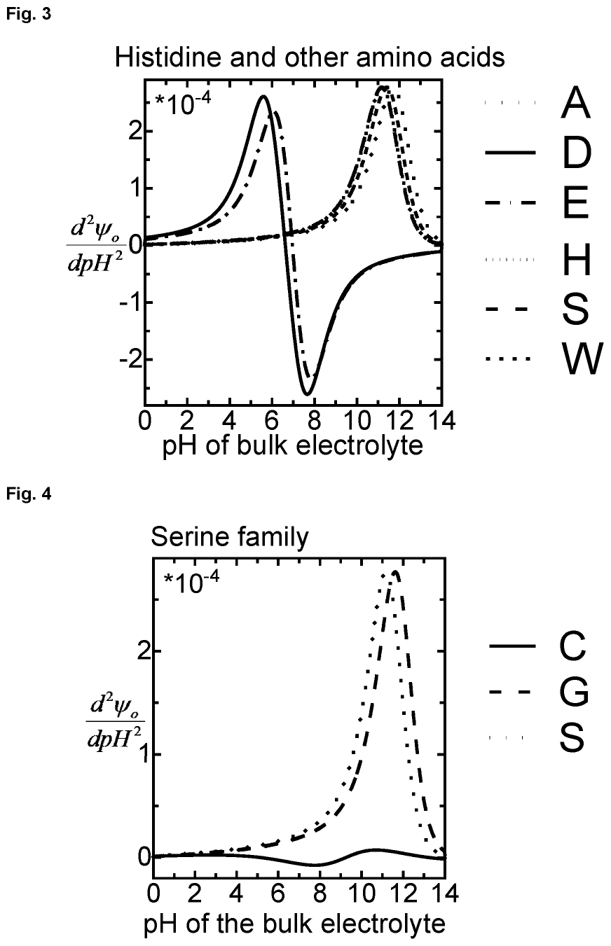

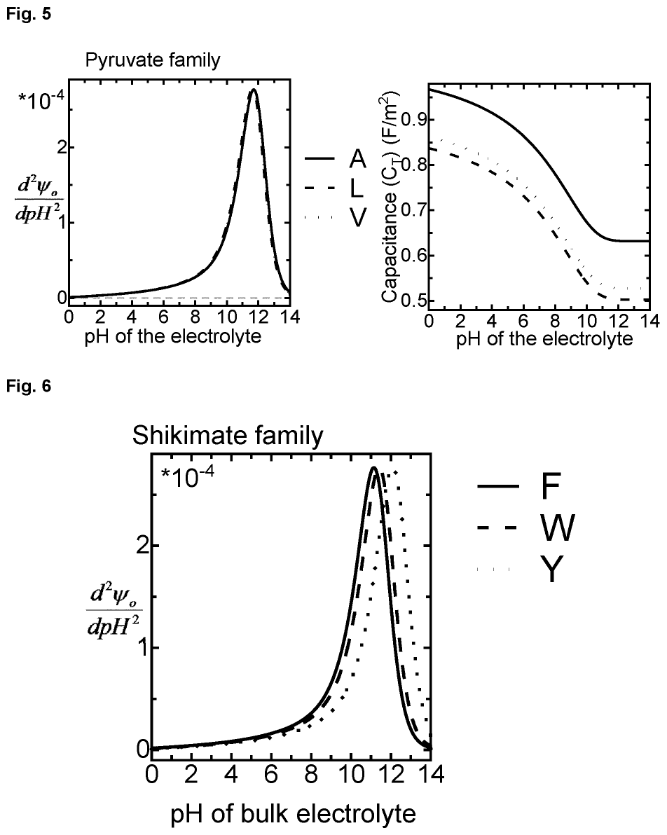

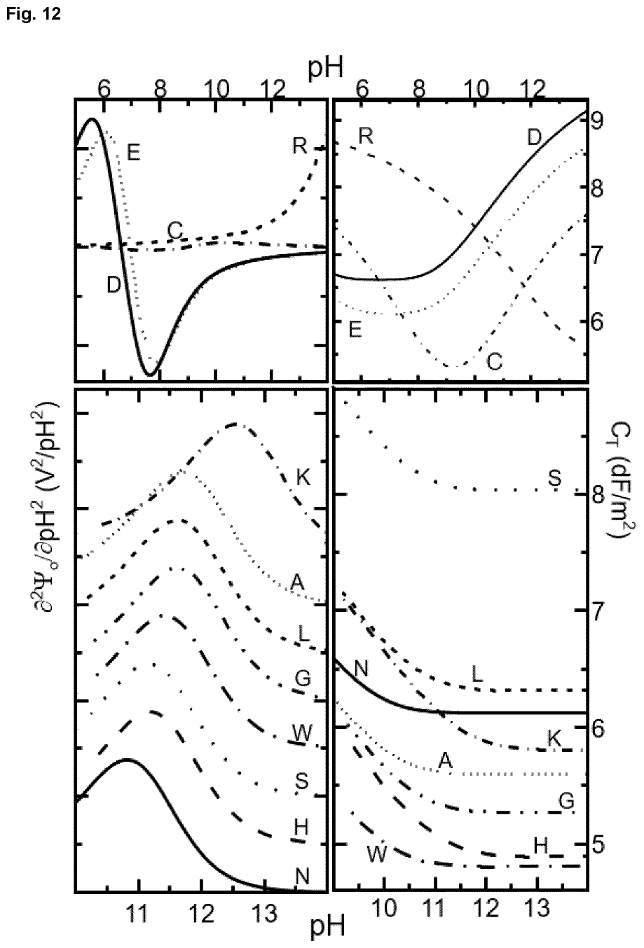

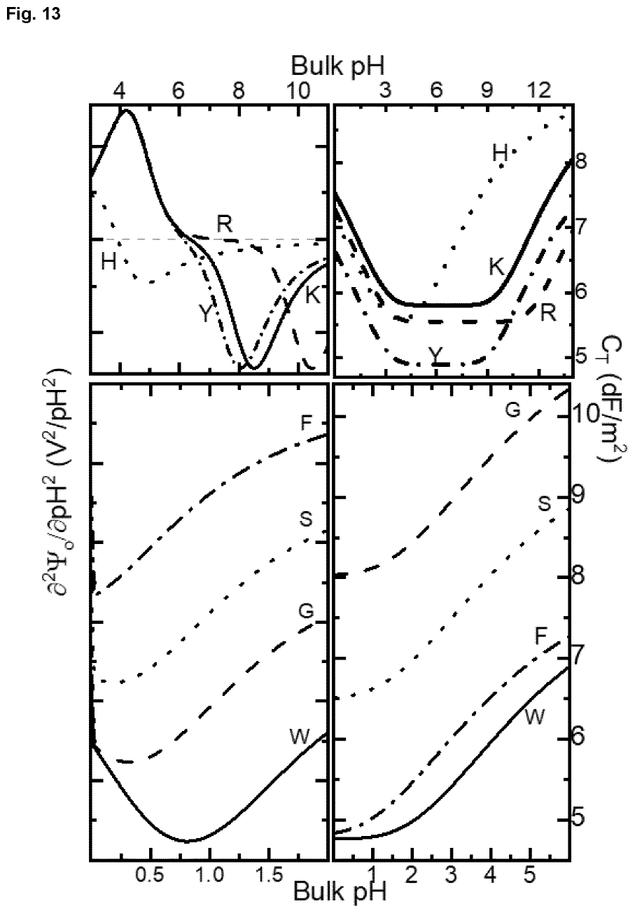

Each curve in FIGS. 1 to 4, in the left-hand diagram of FIG. 5, and in FIG. 6 corresponds to the second derivative of the surface potential with respect to the bulk pH for amino acids immobilized on the sensing surface of a FET sensor by their C-terminus. Similar curves may be obtained for amino acids attached on the sensing surface of a FET sensor by their N-terminus. FIG. 1 shows the curves of the amino acids of the glutamate family (including glutamate (Glu, E), glutamine (Gln, Q), proline (Pro, P) and arginine (Arg, R)), FIG. 2 those of the aspartate family (including aspartate (Asp, D), threonine (Thr, T), asparagine (Asn, N), lysine (Lys, K), methionine (Met, M) and isoleucine (lle, I), FIG. 3 those of histidine (His, H), alanine (Ala, A), aspartate (Asp, D), glutamate (Glu, E), serine (Ser, S) and Tryptophan (Trp, W), FIG. 4 those of the serine family (including serine (Ser, S), glycine (Gly, G) and cysteine (Cys, C)), the left-hand diagram of FIG. 5 those of the pyruvate family (including alanine (Ala, A), valine (Val, V) and leucine (Leu, L)) and FIG. 6 those of the Shikimate family (including tryptophan (Trp, W), phenylalanine (Phe, F) and tyrosine (Tyr, Y)). It can be seen that the curves obtained for most of the amino acids show unique distinguishable features (e.g., positions and amplitudes of maxima and minima). In that case, the fingerprint (which may comprise the curve itself or a digest thereof) thus uniquely identifies the corresponding amino acid. Other curves substantially overlap (e.g., within the pyruvate and Shikimate families) because of highly similar amphoteric properties of the corresponding amino acids. In that case, a curve does not uniquely identify amino acids but uniquely identifies a set (or family) of amino acids. It (or its digest) may thus serve as a fingerprint of the subset of amino acids. To generate a unique fingerprint identifying the concerned amino acids individually, the curves (or the digests thereof) may be combined with additional data. FIGS. 12 and 13 show curves representing the surface potential and the total gate capacitance (C_{T}) of a FET sensor having various amino acids attached by their C- or N-terminus to the FET sensing surface. A unique fingerprint could be composed, for instance, of data derived from (a) the surface potential and/or the gate capacitance of the FET sensor with the amino acid immobilized thereon by its C-terminus, and (b) the surface potential and/or the gate capacitance of the FET sensor with the amino acid immobilized thereon by its N-terminus.

It may be worthwhile noting that similar situations occur with other identification techniques, such as, e.g., mass spectrometry: for amino acids with similar masses, the information obtained by mass spectrometry may reduce the set of possible amino acids but leave an ambiguity. Such ambiguity can often be resolved by taking into account the information of the genetic code producing the protein. Information from the genetic code (when available) can also be combined with the fingerprints obtained according to the present invention.

The fingerprints generated in accordance with the disclosed method may be based on the gate capacitance instead of or in addition to the surface potential Yo. By combining both measurements, a “richer” fingerprint is obtained, potentially reducing the number of residual ambiguities, and increasing the reliability of the identification. Methods for measuring the gate capacitance of an FET sensor having molecules immobilized on its sensing surface are known in the scientific community and need not be detailed herein. The right-hand diagram of FIG. 5 shows the gate capacitance as a function of pH of the amino acids of the pyruvate family. By combining both the gate capacitance and the surface potential Ψ₀ into a fingerprint, these amino acids could be better distinguished.

With particular regard to identification of short peptides, it is noted that temperature modifies the interaction forces among the different bonds, changing their lengths, disentangling weak interactions, and also changing the electro affinities of peptide residues. Therefore, one may improve the acquired fingerprint by measuring the surface potential and/or the gate capacitance not only as a function of acidity but also as a function of temperature. The fingerprint may further be improved by using different FETs and/or different electrolytes. A statistical analysis of the acquired fingerprint may be carried out. A multiplexed device may be used to carry out in parallel various measurements contributing to the fingerprint.

Complex (or “rich”) fingerprints can be implemented using surface potential and gate capacitance measurements at different temperatures and with different (mixtures of) electrolyte.

Reference or known fingerprints are stored in the fingerprint database. While reference fingerprints of individual amino acids can be acquired beforehand, the effort to register fingerprints of short peptides grows exponentially with the length of the peptide chain. It may thus be proposed to introduce an acquired fingerprint into the fingerprint database every time a peptide was successfully identified. Especially in the case of peptides, if an acquired fingerprint does not give rise to a sufficiently close match with a known fingerprint (stored in the fingerprint database), a statistical analysis of the acquired fingerprint may be carried out. This statistical approach can be used in machine learning methods inferring the sequence of a peptide from partial (imperfect) matches of the acquired fingerprint with plural stored fingerprints. In this context, a metric for quantifying the degree of matching between fingerprints, and possibly, a threshold above which a match is considered sufficiently close to conclude that the amino acid or peptide belongs to the species of the stored fingerprint need be defined in concrete implementations of the method.

Advantageous, the FET sensor comprises a graphene FET. It is also an option to use a high-aspect ratio FinFET (e.g., as described in the international application WO 2020/109110 A1). Such a FinFET provides a linear response (improving the dynamic range) as well as good reliability and sensitivity (offering low limits of detection).

The surface potential and/or the gate capacitance are preferably recorded with a signal-to-noise ratio of 30 dB or better. Contributions of chemically active groups (other than the immobilized amino acid or peptide) present on the sensor surface (e.g., silanol groups) shifting the pKa of the immobilized moiety are preferably eliminated. This could be achieved by way of controlled functionalisation of the FET sensor surface, achieving always the same amount (surface density) of unfunctionalized (oxide) groups at the FET-electrolyte interface. Controlled functionalisation of semiconductor-oxide FETs (traditional ISFETs and variations semiconductor-nanowire FETs, FinFETs, chemFETs, immunoFETs, etc.) can be achieved with coatings of alkane, lipid, or polyethylene glycol (PEG) groups (or other equivalent functionalisations) provided that tethering points for immobilization of the amino acid or peptide by the N or C terminal are provided as well.

Undesired contributions to the measurements could also be reduced by using a graphene-based FET or a carbon-nanowire-based FET. A graphene-based FET has no oxide groups on its surface that could contaminate the signal. Immobilization of amino acids and peptides on the surface of a graphene-based FET could be achieved by using a tethering reagent comprising a peri-fused polycyclic aromatic hydrocarbon moiety bonding to the graphene surface of the sensor and a phenyl isothiocyanate functionality to immobilize an amino acid or peptide. An example of such a tethering reagent is phenyl isocyanate-4-(1-pyrene)butyrate (IUPAC name: 4-isothiocyanato-phenyl 4-(3a1,5a1-dihydropyren-1-yl)butanoate), represented by the skeletal formula below:

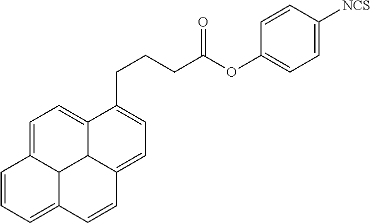

Electrostatic adsorption of the graphene FET sensor could be avoided by back-gating. A graphene FET may have the quantum limit of capacitance, which minimizes noise. Finally, the high conductivity of graphene allows sensing very small amounts of amino acid or peptide.

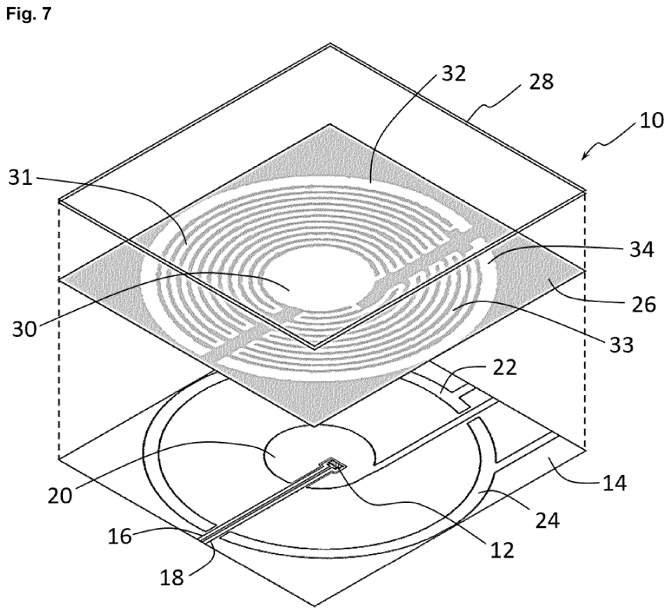

As shown in FIG. 7, the FET sensor 12 may be arranged in a reaction cell 10 configured as an electrochemical transducer translating an applied voltage or current into a change of pH within the reaction cell. The reaction cell may be configured as described in WO 2017/108796 A1, a FET sensor 12 being arranged in the well of the working electrode (hereinafter referred to as the reaction chamber). Measuring the surface potential and/or the gate capacitance of the FET sensor with the amino acid immobilized thereon as a function of pH may then include electrochemically varying the pH in the reaction cell 10. The reaction cell 10 may comprise an insulating substrate 14 carrying the electronic components, such as, the FET 12, the source electrode and its lead 16, the drain electrode and its lead 18, the working electrode and its lead 20, the counter electrode 22, and the reference electrode 24. The height of the reaction cell is defined by an insulating layer 26 (e.g., an epoxy layer) The insulating layer 26 has openings therein defining the reaction chamber 30, a counter electrode chamber 32, a reference electrode chamber 34, a first migration path 31 extending between the reaction chamber and the counter electrode chamber 32 and a second migration path 33 extending between the reaction chamber 30 and the reference electrode chamber 34. The reaction cell 10 further comprises a lid 28 which may be pushed against the insulating layer 26 (or against which the insulating layer 26 may be pushed) in order to close the reaction cell. When the cell is closed, the analyte solution is confined within the volumes of the reaction chamber 30, the counter electrode chamber 32, the reference electrode chamber 34, and the migration paths 31, 33. The volume of the reaction chamber 30 preferably amounts to less than 10 nl or less. The reduced dimensions of the cell may enhance the buffering capacity of the peptides improving the amino acid fingerprints.

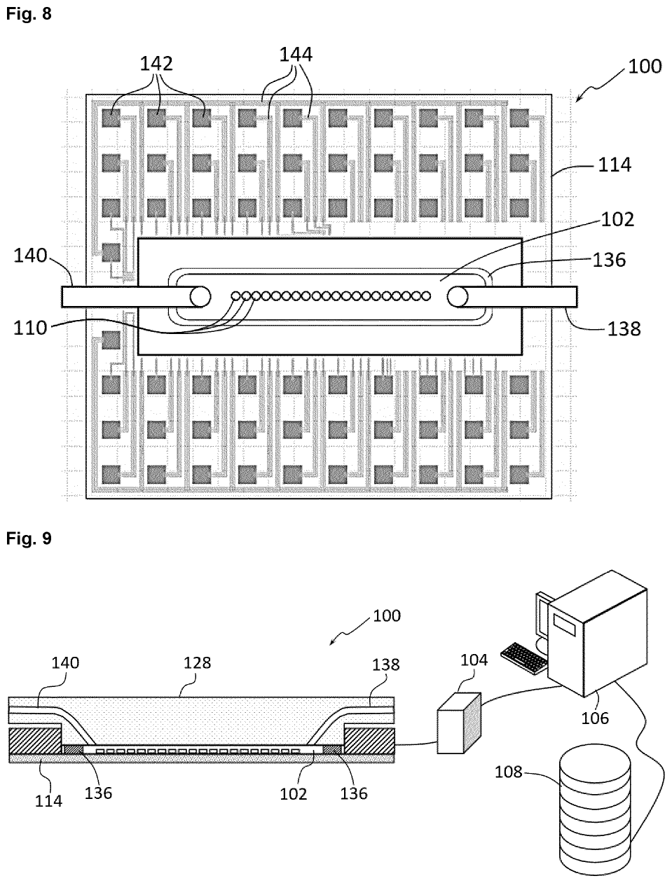

A preferred embodiment of the invention relates to sequencing of peptides. Multiplexed Edman-like degradation and/or enzymatic reactions in miniaturised reaction cells may be used to accurately sequence a peptide. FIGS. 8-10 show a microfluidic device 100 comprising a series of reaction cells 10. Each reaction cell may be configured as show in FIG. 7 and allow regulating acidity and providing addressable control of chemical reactions via pH and/or temperature.

A method for sequencing a peptide may comprise sequentially removing amino acids from the N-or the C-terminus of the peptide, and immobilising the amino acids on the surface of a series of FET sensors, each FET sensor of the series corresponding to a known position in the sequence of removal. For each immobilized amino acid, a fingerprint may be acquired, as described above, and the acquired fingerprint may be looked up in the fingerprint database.

The microfluidic device 100 schematically illustrated in FIGS. 8-10 comprises a microfluidic channel 102, through which an analyte solution may be introduced and evacuated. A series of reaction cells 110 is arranged in the microfluidic channel 102, within a perimeter defined by a gasket 136. In the illustrated embodiment, 20 reaction cells 110 in a linear arrangement are shown, but it will be understood that the number of reaction cells 110 and their geometric arrangement may be changed and adapted to the needs of the intended application. In the shown embodiment, the gasket 136 defines an oblong, generally rectangular perimeter, but also the configuration of the gasket 136 could be adapted to different needs. The microfluidic device 100 comprises an inlet 138 and an outlet 140 opening into the interior of the perimeter defined by the gasket 138 at opposite ends thereof. Droplets of analyte liquid and other liquids (such as, e.g., carrier liquid, rinsing solution, etc.) may be introduced via the inlet 138 and removed through outlet 140. The microfluidic device 100 could comprise several microfluidic channels, which could be arranged in parallel and/or in branched configurations. For sake of simplicity of the drawing, only a basic configuration is shown. In the embodiment of FIGS. 8 to 10, the inlet 138 and the outlet 140 are shown to pass through the common lid 128 of the reaction cells 110 but other arrangements may be possible.

Each reaction cell 110 of the series is configured as an electrochemical transducer translating an applied voltage or current into a change of pH within the the reaction chamber of the reaction cell 110. The reaction chamber of each reaction cell 110 has arranged therein an FET sensor, which comprises a sensing surface functionalised with moieties suited for the sequential removal of an amino acid from a terminus of the peptide (the N-terminus or the C-terminus). The FET sensor could, e.g., be functionalized with 4-chloro phenyl isothiocyanate, or 4-chloro-3(trifluoromethyl)phenyl isothiocyanate.

The microfluidic device 100 further includes a fluid management system for controlling transfer of liquids in the microfluidic channel 102. Such a fluid management system could include one or more droplet generators, pressure and/or vacuum controllers, valves and corresponding controller(s), flow sensors, pressure sensors, actuators (e.g., pneumatic or hydraulic actuators), bubble detectors, reservoirs, sample holders, etc. to prepare and introduce droplets containing the analyte solution into the microfluidic channel 102 with the reaction cells 110.

The transport of amino acids and/or peptides into and/or out of the reaction cell may be achieved with droplets. As used herein, the expression “droplet” may designate a discrete, coherent volume of a liquid (e.g., an electrolyte or a carrier liquid) surrounded by another medium (e.g. gas or another liquid), e.g., with a volume in the range from 50 pl to 5 nl, preferably in the range from 100 pl to 3 nl.

The microfluidic device 100 may include a controller 104 operatively connected to each reaction cell 110 for controlling the pH in its reaction chamber, to the FET sensor of each reaction cell 110 for measuring the surface potential and/or the gate capacitance of the FET sensor to the fluid management system and/or to any other system components. In the illustrated embodiment, the controller 104 may be connected to the working electrode, the counter electrode and the reference electrode of each reaction cell 110. The controller 104 may further be connected to the source and gate terminals of the FET sensor of each reaction cell 110. Preferably, the controller 104 is configured and arranged such that the pH in the reaction chamber of each reaction cell 110 can be controlled individually. If two or more reaction cells 110 should need to be controlled in the same way, e.g., to parallelize manipulations, this would then be possible by programming the controller 104 accordingly.

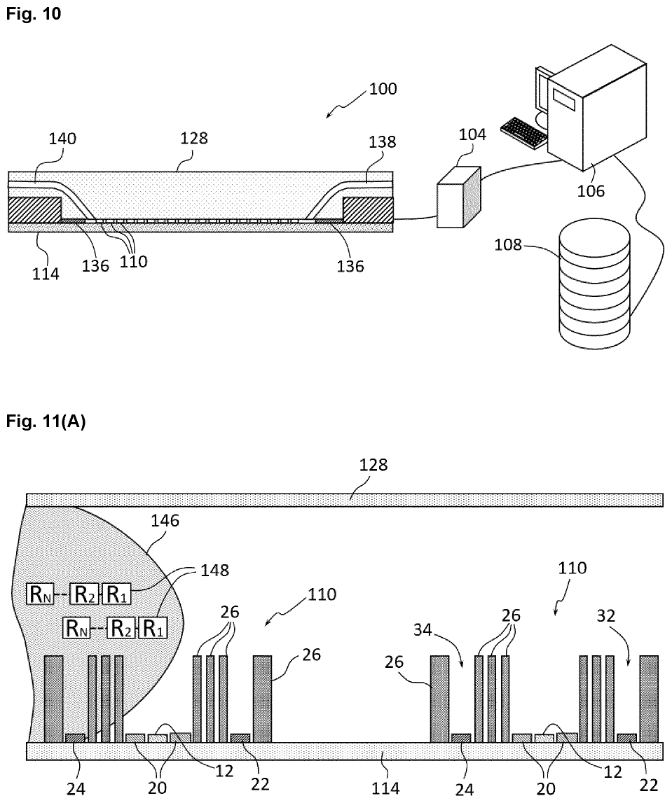

The controller 104 could comprise any type of hardware capable of being interfaced with the various electronic components (actuators, sensors, electrodes, etc. of the microfluidic device). For instance, the controller 104 could comprise or consist of an application-specific integrated circuit (ASIC), a system on a chip (SoC), a programmable logic device (PLD), an erasable programmable logic device (EPLD), a programmable logic array (PLA), a field-programmable gate array (FPGA), a generic microprocessor, a combination of the above and/or other hardware system adequately programmed for the task. The controller 104 may include one or more analog-to-digital converters (ADC) and/or one or more digital-to-analog converters (DAC). In FIGS. 9 and 10, the controller 104 is schematically shown as a boxed unit connected to a workstation 106 and a database server 108. The reader will understand that this is only a schematic illustration to explain the principles and aspects of the invention and its embodiments.

The database server 108 may host the fingerprint database. The workstation 106 may have software (e.g., an application providing a graphical user interface of the microfluidic device 100) installed thereon that is configured to log the measurements made with the microfluidic device 100, in particular, to record the raw data, and to compute the fingerprints from the measurements. The software may further be configured to look up the fingerprints stored in the fingerprint database. The software could include an artificial intelligence module configured, e.g., to search for similarities between not readily identifiable fingerprints and the fingerprints stored in the fingerprint database and, taking into account the analysis of similarities, to output (and, e.g., to display) one or more candidate amino acids or candidate peptide sequences that could have occasioned the not readily identifiable fingerprints. In the present context, a “not readily identifiable fingerprint” may be an acquired fingerprint that does not give rise to a (sufficiently close) match with a known fingerprint (stored in the fingerprint database). In a practical implementation, this may mean that the degrees of similarity (measured according to the similarity metric used by the algorithm) between the acquired fingerprint and the stored fingerprints remain below the detection (or identification) threshold associated to a match (or a close match). The lookup software may include an autonomous learning algorithm that can be used during a training phase and, optionally, during regular use in order to increment the fingerprint database and improve the lookup module.

The reaction cells 110 are arranged on a common substrate 114, e.g., a printed circuit board. The common substrate 114 may be that of a SOI (silicon-on-insulator) wafer in and on which the electronic components and the insulating layer patterning the chambers of the reaction cells 110 (see insulating layer 26 in FIG. 7) are formed. The microfluidic device 100 further comprises a lid 128 common to all reaction cells 110. An actuator (not shown) is provided to open and close the reaction cells 110 by moving the substrate 114 and the lid relative to each other. The actuator may be a pneumatic actuator under the control of the controller 104. The actuator may push the common substrate 114 against the lid 128 (which could, e.g., be made of glass or plastic if transparency of the lid is desired), whereby the lid 128 is put into contact with the insulating layer and the reaction cells 110 are closed (see FIG. 10). The reaction cells 110 are opened by moving the substrate 114 away from the lid (see FIG. 9).

As shown in FIG. 8, contact pads 142 may be arranged on the substrate 114 offside the microfluidic channel 102. The contact pads 142 may be connected to the electrodes and terminals of the reaction cells 110 by conductive traces 144 and serve to interface the reaction cells 110 with the controller 104.

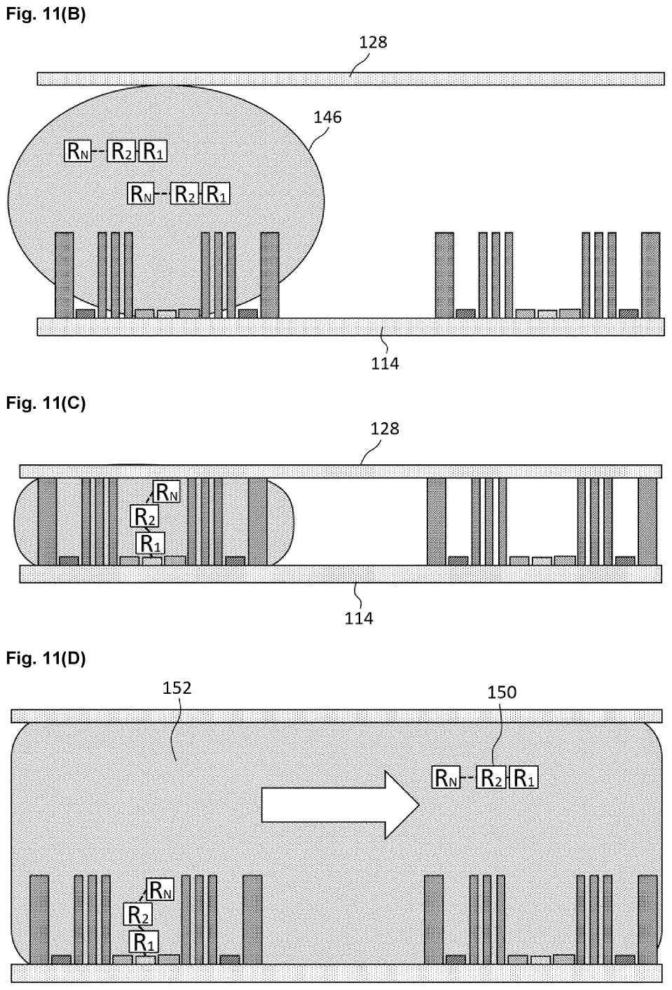

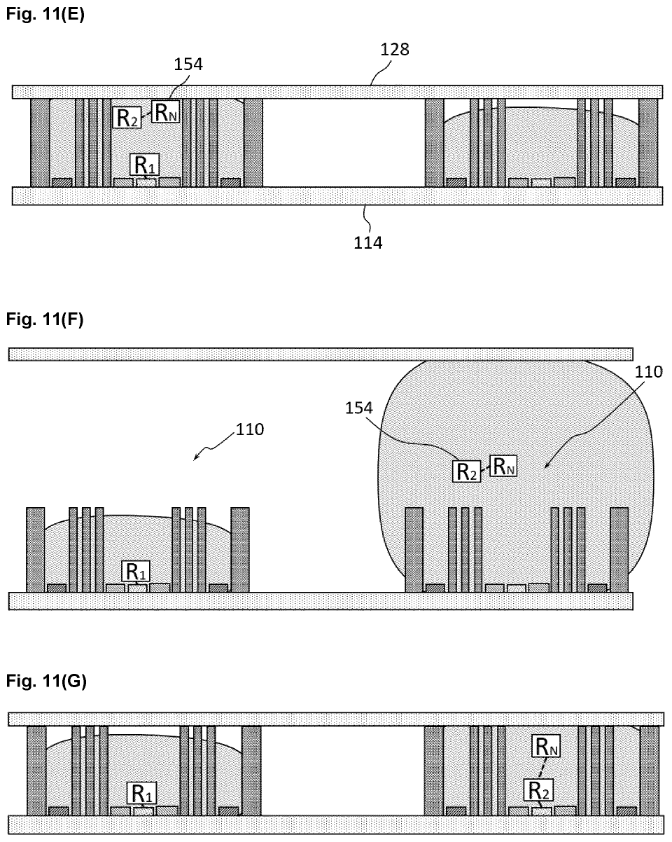

Sequencing of a peptide may be carried out with the microfluidic device 100 as follows. As mentioned before, the sensing surfaces of the FET sensors are functionalized with moieties suited for the sequential removal of an amino acid from a terminus of the peptide. A droplet of analyte solution 146, containing the peptide 148 to be sequenced is introduced into the reaction cell 110 closest to the inlet 138 (hereinafter: the first reaction cell) as shown in FIG. 11 (A) and FIG. 11 (B). The peptide 148 to be sequenced comprises several (possibly unknown) amino acids R₁, R₂, . . . , R_{N}. The peptide 148 to be sequenced may be provided in a highly pure form. It may be obtained by any technique capable of separating peptide species, e.g., high-performance liquid chromatography (HPLC).

In the following description, it will be assumed that the functionalization has been effected with PITC molecules, which can be used for classical Edman degradation. It shall be understood, however, that other types of functionalization may be used as well. The steps of the sequencing process include:
(a) The reaction cells 110 are closed by pushing the lid 128 against the substrate 114. The pH in the reaction chamber of the first reaction cell is then adjusted such that one terminus (R1 in FIGS. 11 (A)-(C)) of the peptide 148 reacts with the tethering moiety (not shown). If the FET is functionalised with PITC, the peptide bonds to the sensing surface by its N-terminus in basic conditions (see FIG. 11 (C)).
(b) After the time of incubation, the reaction cells 110 are opened and one or more droplets of a rinsing liquid152 are introduced into the microfluidic channel. The rinsing liquid 152 removes (washes away) non-attached peptide 150 from the first reaction cell (FIG. 11 (D)).
(c) The reaction cells 110 are closed and the pH in the reaction chamber of the first reaction cell is adjusted to separate the terminal amino acid R1 (i.e., the amino acid directly attached to the tethering moiety) from the peptide. In the illustrated case, this is the N-terminal amino acid and the cleavage occurs under acidic conditions. The terminal amino acid remains attached to the sensing surface of the FET sensor in the first reaction cell, while the cleaved peptide 154 (without the removed terminal amino acid R₁) is released into the electrolyte (FIG. 11 (E)).
(d) The reaction cells 110 are opened, and one or more droplets of electrolyte are introduced to carry over the cleaved peptide 154 into the second reaction cell (the neighbour of the first reaction cell) (FIG. 11 (F)). The terminal amino acid R₁ remains attached to the sensing surface of the FET sensor in the first reaction cell and may be identified as set forth above.

The above-described steps are then repeated in the second reaction cell so that the second amino acid of the initial peptide is attached on the sensing surface of the FET sensor in the second reaction cell (FIG. 11 (G)). The remainder of the peptide (having two amino acids removed from its N-terminal) is carried over into the next reaction cell in the series. The same steps are then repeated again in the third, fourth, fifth, etc. reaction cells, such that the amino acids are immobilized in the reaction cells in the order of the peptide sequence. In the last reaction cell, the cycle may be stopped when the peptide carried over from the penultimate reaction cell has been attached to the FET sensor. Alternatively, it is possible to cleave the remaining peptide and keep the terminal amino acid attached to the FET sensor of the last reaction cell.

It is worthwhile noting that instead of using individual reaction cells for each sequencing step, one could also use groups of reaction cells by appropriate control of the droplets introduced into the microfluidic channel and the reactions in the reaction cells. One could thereby arrive at a first group of reaction cells having immobilized therein the first amino acid of the peptide sequence, a second group of reaction cells having immobilized therein the second amino acid of the peptide sequence, etc. The groups of reaction cells could have the same number of group members but it would also be possible to define the groups with different numbers of group members. This may be useful for compensating losses in peptide concentration from one sequencing step to the next: one or more of the upstream sequencing steps could be carried out in parallel in plural reaction cells in order to make more cleaved peptide available in the downstream sequencing steps.

It should also be noted that the microfluidic device may be equipped with one or more heating elements and/or cooling elements (e.g., Peltier elements) to adjust the temperature of the reaction cells individually or globally.

The amino acids or peptides attached to the FET sensors may be identified as set forth above (FIG. 11 (F)). The fingerprints acquired for each reaction cell permit to identify the immobilized molecules. If an unambiguous identification should not be possible using the acquired fingerprints only, further analyses may be carried out. Also information from the genetic code (when available) can also be combined with the fingerprints obtained in each reaction cell.

While specific embodiments have been described herein in detail, those skilled in the art will appreciate that various modifications and alternatives to those details could be developed in light of the overall teachings of the disclosure. Accordingly, the particular arrangements disclosed are meant to be illustrative only and not limiting as to the scope of the invention, which is to be given the full breadth of the appended claims and any and all equivalents thereof.

1. A method for identifying an amino acid, comprising:
immobilising the amino acid on the surface of a FET sensor;
acquiring a fingerprint of the immobilized amino acid, the acquisition of the fingerprint including measuring at least one of the surface potential and the gate capacitance of the FET sensor with the amino acid immobilized thereon as a function of pH,
looking up the acquired fingerprint in a fingerprint database.

2. The method as claimed in claim 1, wherein the FET sensor is arranged in a reaction chamber of a reaction cell configured as an electrochemical transducer translating an applied voltage or current into a change of pH within the reaction chamber of the reaction cell, and wherein measuring the at least one of the surface potential and the gate capacitance of the FET sensor with the amino acid immobilized thereon as a function of pH includes electrochemically varying the pH in the reaction chamber of the reaction cell.

3. The method as claimed in claim 2, wherein during the measurement of the at least one of the surface potential and the gate capacitance as a function of pH, the reaction cell is closed, the volume of the reaction chamber of the reaction cell being less than 10 nl.

4. The method as claimed in claim 1, wherein the acquisition of the fingerprint includes measuring both the surface potential and the gate capacitance of the FET sensor with the amino acid immobilized thereon as a function of pH.

5. The method as claimed in claim 1, wherein the acquisition of the fingerprint includes measuring the at least one of the surface potential and the gate capacitance of the FET sensor with the amino acid immobilized thereon as a function of pH at constant temperature.

6. The method as claimed in claim 1, wherein the acquisition of the fingerprint includes measuring the at least one of the surface potential and the gate capacitance of the FET sensor with the amino acid immobilized thereon as a function of pH at at least two temperatures, the temperature being maintained constant during each measurement of the at least one of the surface potential and the gate capacitance as a function of pH.

7. The method as claimed in claim 1, wherein the acquisition of the fingerprint comprises determining the second derivative of the surface potential with respect to pH.

8. The method as claimed in claim 1, wherein the acquisition of the fingerprint comprises determining the first derivative of the gate capacitance with respect to pH.

9. The method as claimed in claim 1, wherein the amino acid is immobilized on the surface of the FET sensor with a PITC homologue reagent.

10. The method as claimed in claim 1, wherein the FET sensor comprises a graphene FET sensor.

11. The method as claimed in claim 1, wherein the FET sensor comprises a dielectric layer made of a high-K dielectric oxide, e.g., Al₂O₃, HfO₂, or TiO₂.

12. The method as claimed in claim 1, wherein the FET sensor includes a FinFET.

13. The method as claimed in claim 1, wherein the fingerprint is acquired with the FET sensor immersed in a first electrolyte and wherein a further fingerprint is acquired with the FET sensor immersed in a second, different electrolyte.

14. A method for sequencing a peptide, comprising:
sequentially removing amino acids from a terminus of the peptide, the terminus being the N-terminus or the C-terminus of the peptide;
immobilising the amino acids on the surface of a series of FET sensors, each FET sensor of the series corresponding to a known position in the sequence of removal;
acquiring a fingerprint of each immobilized amino acid, the acquisition of the fingerprint including measuring at least one of the surface potential and the gate capacitance of the respective FET sensor with the amino acid immobilized thereon as a function of pH, and for each FET sensor, looking up the acquired fingerprint in a fingerprint database.

15. A device for recording a fingerprint of an analyte, comprising
a reaction cell configured as an electrochemical transducer translating an applied voltage or current into a change of pH within a reaction chamber of the reaction cell, the reaction chamber of the reaction cell having arranged therein an FET sensor, the FET sensor comprising a sensing surface for immobilizing the analyte thereon;
a controller operatively connected to the reaction cell for controlling the pH in the reaction chamber and to the FET sensor for measuring at least one of the surface potential and the gate capacitance of the FET sensor; the controller being configured to execute a fingerprint acquisition routine, which includes recording the at least one of the surface potential and the gate capacitance of the FET sensor while increasing or decreasing the pH in the reaction chamber of the reaction cell.

16. The device as claimed in claim 15, wherein the reaction cell comprises a heating element and wherein the controller is operatively connected to the heating element for controlling the temperature in the reaction chamber of the reaction cell.

17. The device as claimed in claim 15, wherein the surface of the FET sensor is functionalised with a PITC homologue reagent.

18. The device as claimed in claim 15, wherein the FET sensor comprises a graphene FET sensor.

19. The device as claimed in claim 15, wherein the FET sensor comprises a dielectric layer made of a high-K dielectric oxide, e.g., Al₂O₃, HfO₂, or TiO₂.

20. The device as claimed in claim 15, wherein the FET sensor includes a FinFET.
